SEQUENCE LISTING (22 sequences)

SEQ ID NO: 1 - DNA, 2805 bp, Nicotiana tabacum
gaattcggcacgagggaaactatatacagagagacataatttgaagaaacgctgaaagtgattatgttataatcacagtatatgtcagtagcctctaatagtcactgttggggttctcatcgcagctttcttcctagctaagcagtactcacaatataatggccactactaaacagaaagtgacggcaccttctccttctccttcttcttcgactgcttcttgctgtccttccacttctatcctccgtcgtgaggcaacagcggccattgcagtcgtgggtgacggcctgcagaattggaccaacatcccctccgtcgacgagaagcagaaaaagacggcctcatcagctctagcgtcattgccaaccactgaacctctttccaccaatacctctaccaaaggtatccaaatcatgacaagggctcaaacctgccatcctttggaccctttatctgctgctgagatctcagtggctgtggcaactgttagagctgccggtgaaacacctgaggtcagagatgggatgcgatttattgaggtggttctggtagaaccagataaaagtgtagttgcattggcagatgcatatttcttcccaccttttcagtcatcattgatgccgagaaccaaaggaggatctcagattcctactaagcttcctccaaggagagctaggcttattgtttacaataagaaaacaaatgagacaagcatttggattgttgagctaaacgaagtacatgctgctgctcgaggtggacatcacaggggaaaagtcatcgcatccaatgttgtccctgatgttcagccacccatagatgctcaagagtatgctgaatgtgaagctgtggtgaaaagttatcctccctttcgagacgcaatgaggagaaggggtattgatgacttggatcttgtgatggttgacccttggtgtgttggttatcatagtgaggctgatgctcctagccgcaggctcgcgaaaccacttgtattctgcaggacagagagtgactgcccaatggaaaatggatatgcaagaccagttgaaggaatatatgtgcttgttgatgtacaaaacatgaagattatagaatttgaagaccgaaaacttgtaccattacctccagttgacccactgaggaactacactgctggtgagacaagaggaggggttgatcgaagtgatgtgaaacccctacatattattcagcctgagggtccaagctttcgtatcagtggaaactacgtagagtggcagaagtggaactttcggattggtttcacccctagagagggtttagttatacactctgtggcgtatcttgatggtagcagaggtcgtagaccaatagcacataggttgagttttgtagagatggttgtcccctatggagatccaaatgatccacattataggaagaatgcatttgatgcaggagaagatggccttggaaagaatgctcattcactgaagaggggatgtgattgtttagggtacataaagtactttgatgcccatttcacaaactttaccggaggagttgaaacgactgaaaattgtgtatgcttgcatgaagaagatcacggaatgctttggaagcatcaagattggagaactggccttgctgaagttagacggtctaggcgactaacagtgtcttttgtttgtacagtggccaattatgaatatgcattctactggcatttctaccaggatggaaaaattgaagcggaagtcaaactcactggaattcttagtttgggagcattgcaacctggagaatatcgcaaatatggtaccacaattttaccaggtttgtatgcaccagttcatcaacacttctttgttgcacgaatgaatatggcagttgattgtaagccaggagaagcacacaatcaggttgttgaagtaaatgtcaaagttgaagaacctggcaaggaaaatgttcataataatgcattctatgctgaagaaacattgcttaggtctgaattgcaagcaatgcgtgattgtgatccattctctgctcgtcattggattgttaggaacacaagaacagtaaatagaacaggacagctaacagggtacaagctggtacctggtccaaactgtttgccactggctggtcctgaggcgaaatttttgagaagagctgcatttctgaagcacaatctatgggttacacaatatgcacctggagaagattttccaggaggagaagttccctaatcaaaatccccgtgttggcgagggattagcttcttgggtcaagcaagaccggcctctggaagaaagtgatattgttctctggtatatttttggaatcacacatgttcctcggttggaagactggcctgttatgccagtagaacacattggttttgtgctacagccacatggatactttaactgctctccggctgttgatgtccctccgccctttgcatgcgactcagaaagcagagacagtgatgttactgaaactagtgtagcaaagtccactgccactagcttgctggccaagctttgaatgtttcgtttatcctaacatgagtcctcctcgatcgcctatttacctacggataccaaacttcatttttcttttgatagagtattgaattagttggttcaggaacatggttttgactagtcgcatatatggcacgtttaagcaaagcaagtccctttgtgtattgatcgtgaataaagcatgttatagggaaaaactcataaatgtcgatctttggtaactctcggtcgttgcatttcatttaaaaaaaaaaaaaaaaaaaaaaaactcgag

SEQ ID NO: 2 - protein, 790 aa, Nicotiana tabacum
MATTKQKVTAPSPSPSSSTASCCPSTSILRREATAAIAVVGDGLQNWTNIPSVDEKQKKTASSALASLPTTEPLSTNTSTKGIQIMTRAQTCHPLDPLSAAEISVAVATVRAAGETPEVRDGMRFIEVVLVEPDKSVVALADAYFFPPFQSSLMPRTKGGSQIPTKLPPRRARLIVYNKKTNETSIWIVELNEVHAAARGGHHRGKVIASNVVPDVQPPIDAQEYAECEAVVKSYPPFRDAMRRRGIDDLDLVMVDPWCVGYHSEADAPSRRLAKPLVFCRTESDCPMENGYARPVEGIYVLVDVQNMKIIEFEDRKLVPLPPVDPLRNYTAGETRGGVDRSDVKPLHIIQPEGPSFRISGNYVEWQKWNFRIGFTPREGLVIHSVAYLDGSRGRRPIAHRLSFVEMVVPYGDPNDPHYRKNAFDAGEDGLGKNAHSLKRGCDCLGYIKYFDAHFTNFTGGVETTENCVCLHEEDHGMLWKHQDWRTGLAEVRRSRRLTVSFVCTVANYEYAFYWHFYQDGKIEAEVKLTGILSLGALQPGEYRKYGTTILPGLYAPVHQHFFVARMNMAVDCKPGEAHNQVVEVNVKVEEPGKENVHNNAFYAEETLLRSELQAMRDCDPFSARHWIVRNTRTVNRTGQLTGYKLVPGPNCLPLAGPEAKFLRRAAFLKHNLWVTQYAPGEDFPGGEFPNQNPRVGEGLASWVKQDRPLEESDIVLWYIFGITHVPRLEDWPVMPVEHIGFVLQPHGYFNCSPAVDVPPPFACDSESRDSDVTETSVAKSTATSLLAKL

SEQ ID NO: 3 - protein, 684 aa, Arthrobacter globiformis
MTLQTTPSTPLVQDPPVPATLVHAAAQHPLEQLSAEEIHEARRILAEAGLVGESTRFAYLGLIEPPKTTRQGDVTGAARLVRAMLWDAAQSRSLDVRLSLATGLVVDRRELNPEADGQLPVLLEEFGIIEDILSEDPQWNAALTARGLTPAQVRVAPLSAGVFEYGNEEGKRLLRGLGFRQDHPADHPWAHPIDGLVAFVDVENRRVNHLIDDGPVPVPEVNGNYTDPAIRGELRTDLLPIEIMQPEGPSFTLEGNHLSWAGWDLRVGFDAREGLVLHQLHHSHKGRRRPVIHRASISEMVVPYGDPSPYRSWQNYFDSGEYLVGRDANSLRLGCDCLGDITYMSPVVADDFGNPRTIENGICIHEEDAGILWKHTDEWAGSDEVRRNRRLVVSFFTTVGNYDYGFYWYLYLDGTIEFEAKATGIVFTAALPDKDYAYASEIAPGLGAPYHQHLFSARLDMMIDGDANRVEELDLVRLPKGPGNPHGNAFTQKRTLLARESEAVRDADGAKGRVWHISNPDSLNHLGHPVGYTLYPEGNPTLAMADDSSIASRAAFARHHLWVTRHAEEELYAAGDFVNQHPGGAVLPAYVAQDRDIDGQDLVVWHSFGLTHFPRPEDWPIMPVDTTGFTLKPHGFFDENPTLNVPSSAAGHCGTGSEREHAAPGGTAVGHSGPDTGGQGHCGH

SEQ ID NO: 4 - protein, 757 aa, Escherichia coli
MGSPSLYSARKTTLALAVALSFAWQAPVFAHGGEAHMVPMDKTLKEFGADVQWDDYAQLFTLIKDGAYVKVKPGAQTAIVNGQPLALQVPVVMKDNKAWVSDTFINDVFQSGLDQTFQVEKRPHPLNALTADEIKQAVEIVKASADFKPNTRFTEISLLPPDKEAVWAFALENKPVDQPRKADVIMLDGKHIIEAVVDLQNNKLLSWQPIKDAHGMVLLDDFASVQNIINNSEEFAAAVKKRGITDAKKVITTPLTVGYFDGKDGLKQDARLLKVISYLDVGDGNYWAHPIENLVAVVDLEQKKIVKIEEGPVVPVPMTARPFDGRDRVAPAVKPMQIIEPEGKNYTITGDMIHWRNWDFHLSMNSRVGPMISTVTYNDNGTKRKVMYEGSLGGMIVPYGDPDIGWYFKAYLDSGDYGMGTLTSPIARGKDAPSNAVLLNETIADYTGVPMEIPRAIAVFERYAGPEYKHQEMGQPNVSTERRELVVRWISTVGNYDYIFDWIFHENGTIGIDAGATGIEAVKGVKAKTMHDETAKDDTRYGTLIDHNIVGTTHQHIYNFRLDLDVDGENNSLVAMDPVVKPNTAGGPRTSTMQVNQYNIGNEQDAAQKFDPGTIRLLSNPNKENRMGNPVSYQIIPYAGGTHPVAKGAQFAPDEWIYHRLSFMDKQLWVTRYHPGERFPEGKYPNRSTHDTGLGQYSKDNESLDNTDAVVWMTTGTTHVARAEEWPIMPTEWVHTLLKPWNFFDETPTLGALKKDK

SEQ ID NO: 5 - protein, 655 aa, Hansenula polymorpha
  MOD_RES (388): Any amino acid
PARPAHPLDPLSTAEIKAATNTVKSYFAGKKISFNTVTLREPARKAYIQWKEQGGPLPPRLAYYVILEAGKPGVKEGLVDLASLSVIETRALETVQPILTVEDLCSTEEVIRNDPAVIEQCVLSGIPANEMHKVYCDPWTIGYDERWGTGKRLQQALVYYRSDEDDSQYSHPLDFCPIVDTEEKKVIFIDIPNRRRKVSKHKHANFYPKHMIEKVGAMRPEAPPINVTQPEGVSFKMTGNVMEWSNFKFHIGFNYREGIVLSDVSYNDHGNVRPIFHRISLSEMIVPYGSPEFPHQRKHALDIGEYGAGYMTNPLSLGCDCKGVIHYLDAHFSDRAGDPITVKNAVCIHEEDDGLLFKHSDFRDNFATSLVTRATKLVVSQIFTAANXEYCLYWVFMQDGAIRLDIRLTGILNTYILGDDEEAGPWGTRVYPNVNAHNHQHLFSLRIDPRIDGDGNSAAACDAKSSPYPLGSPENMYGNAFYSEKTTFKTVKDSLTNYESATGRSWDIFNPNKVNPYSGKPPSYKLVSTQCPPLLAKEGSLVAKRAPWASHSVNVVPYKDNRLYPSGDHVPQWSGDGVRGMREWIGDGSENIDNTDILFFHTFGITHFPAPEDFPLMPAEPITLMLRPRHFFTENPGLDIQPSYAMTTSEAKRAV

SEQ ID NO: 6 - protein, 674 aa, Pisum sativum
MASTTTMKLALFSVLTLLSFHAVVSVTPLHVQHPLDPLTKEEFLAVQTIVQNKYPISKNKLAFHYIGLDDPEKDHVLRYETHPTLVSIPRKSFVVAIINSQTHEILIDLRIRSIVSDNIHNGYGFPILSVDEQSLAIELPLKYPPFIDSVKKRGLNLSEIVCSSFTMGWFGEEKNVRTVRLDCFMKESTVNIYVRPITGITIVADLDLMKIVEYHDRDIEAVPTAENTEYQVSKQSPPFGPKQHSLTSHQPQGPGFQIEGHSVSWANWKFHIGFDVRAGIVISLASIYDLEKHKSRRVLYKGYISELFVPYQDPTEEFYFKTFFDSGEFGFGLSTVSLIPNRDCPPHAQFIDTYIHSANGTPILLKNAICVFEQYGNIMWRHTENGIPNESIEESRTEVNLIVRTIVTVGNYDNVIDWEFKASGSIKPAIALSGILEIKGTNIKHKDEIKEDLHGKLVSANSIGIYHDHFYIYYLDFDIDGTHNSFEKTSLKTVRIKDGSSKRKSYWTTETQTAKTESDAKITIGLAPAELVVVNPNIKTAVGNEVGYRLIPAIPAHPLLTEDDYPQIRGAFTNYNVWVTAYNRTEKWAGGLYVDHSRGDDTLAVWTKQNREIVNKDIVMWHVVGIHHVPAQEDFPIMPLLSTSFELRPTNFFERNPVLKTLSPRDVAWPGCSN

SEQ ID NO: 7 - DNA, 20 bp, Artificial Sequence
  Description of Artificial Sequence Synthetic oligonucleotide
gtngtnccntayggngaycc

SEQ ID NO: 8 - DNA, 20 bp, Artificial Sequence
  Description of Artificial Sequence Synthetic oligonucleotide
ggcatnaynggccartcytc

SEQ ID NO: 9 - DNA, 22 bp, Artificial Sequence
  Description of Artificial Sequence Synthetic primer
ttcacaaactttacgggaggag

SEQ ID NO: 10 - DNA, 21 bp, Artificial Sequence
  Description of Artificial Sequence Synthetic primer
tcgagcgagtatcaaagaaat

SEQ ID NO: 11 - DNA, 24 bp, Artificial Sequence
  Description of Artificial Sequence Synthetic primer
cgtgactgtgatccattctctgct

SEQ ID NO: 12 - DNA, 24 bp, Artificial Sequence
  Description of Artificial Sequence Synthetic primer
tgtaacccatagattgtgcttcag

SEQ ID NO: 13 - DNA, 25 bp, Artificial Sequence
  Description of Artificial Sequence Synthetic primer
tccatggccactactaaacagaaag

SEQ ID NO: 14 - DNA, 24 bp, Artificial Sequence
  Description of Artificial Sequence Synthetic primer
taacaggccagtcttccaaccgag

SEQ ID NO: 15 - DNA, 22 bp, Artificial Sequence
  Description of Artificial Sequence Synthetic primer
tcaaaatccccgtgttggcgag

SEQ ID NO: 16 - DNA, 30 bp, Artificial Sequence
  Description of Artificial Sequence Synthetic primer
ggatccccatggccactactaaacagaaag

SEQ ID NO: 17 - DNA, 22 bp, Artificial Sequence
  Description of Artificial Sequence Synthetic primer
tggtagaggtattggtggaaag

SEQ ID NO: 18 - protein, 329 aa, Nicotiana tabacum
VVPYGDPNDPHYRKNAFDAGEDGLGKNAHSLKRGCDCLGYIKYFDAHFTNFTGGVETTENCVCLHEEDHGMLWKHQDWRTGLAEVRRSRRLTVSFVCTVANYEYAFYWHFYQDGKIEAEVKLTGILSLGALQPGEYRKYGTTILPGLYAPVEQHFFVARMNMAVDCKPGEAENQVVEVNVKVEEPGKENVHNNAFYAEETLLRSELQAMRDCDPTSARHWIVRNTRTVNRTGQLTGYKLVPGPNCLPLAGPEAKFLRRAAFLKHNLWVTQYAPGEEFPGGEFPNQNPRVGEGLASWVKQDRPLEESDIVLWYIFGITEVPRLEDWPIMP

SEQ ID NO: 19 - protein, 776 aa, Arabidopsis thaliana
MASASKKTSACPHHGGSLPPPKLVSAAPDTVAVWSDADDQRASKVSLESVIRPVDSFPDNTAKKPANKGISVMPRTETKHPLDPLSAAEISVAVATVRAAGANPEVRDGMRFIEVASVEPDKQVVALADAYFFPPFQPSLLPRTKSGPVIPMKLPPRRARLVVYNQKSNETSVWIVALSEVHAVTRGGHHRGRVVSSQVIPDVQPPMDAAEYAECEAIVKDFPPFIEAMKRRGIEDMDLVMVDPWCVGYHSEADAPSRRLAKPLIYCRTDSDSPMENGYARPVEGIYVLVDMQNMVVIEFEDRKFVPLPPPDPLRNYTPGESRGGVDRSDVKPLQIIQPEGPSFRVRGYFVEWQKWNFRIGFTPREGLVIHSVAYVDGSRGRRPVAHRLSFVEMVVPYGDPNEPHYRKNAFDAGEDGLGKNAHSLKKGCDCLGSIKYFDAHFTNFTGGVETIENCVCLHEEDHGILWKHQDWRTGLAEVRRSRRLTVSFLCTVANYEYGFYWHFYQDGKIEAEVKLTGILSLGALQPGETRKYGTTIAPGLYAPVHQHFFIARMDMSVDCKPAEAFNQVVEVNVRVDERGENNVHNNAFYAEEKLLKSEAVAMRDCDPLSARHWIVRNTRTVNRTGQLTGYKLVPGSNCLPLARPEAKFLRRAAFLKHNLWVTRYAPDEKFPGGEFPNQNPRAGEGLATWVKQNRSLEESDVVLWYVFGITHVPRLEDWPVMPVEHIGFTLMPHGFFNCSPAVDVPPNPCELETKESEVKEVVAPKALQTGLLSKL

SEQ ID NO: 20 - protein, 638 aa, Arthrobacter globiformis
MTPSTIQTASPFRLASAGEISEVQGILRTAGLLGPEKRIAYLGVLDPARGAGSEAEDRRFRVFIHDVSGARPQEVTVSVTNGTVISAVELDTAATGELPVLEEEFEVVEQLLATDERWLKALAARNLDVSKVRVAPLSAGVFEYAEERGRRILRGLAFVQDFPEDSAWAHPVDGLVAYVDVVSKEVTRVIDTGVFPVPAEHGNYTDPELTGPLRTTQKPISITQPEGPSFTVTGGNHIEWEKWSLDVGFDVREGVVLHNIAFRDGDRLRPIINRASLAEMVVPYGDPSPIRSWQNYFDTGEYLVGQYANSLELGCDCLGDITYLSPVISDAFGNPREIRNGICMHEEDWGILAKHSDLWSGINYTRRNRRMVISFFTTIGNYEYGFYWYLYLDGTIEFEAKATGVVFTSAFPEGGSDNISQLAPGLGAPFHQHIFSARLDMAIDGFTNRVEEEDVVRQTMGPGNERGNAFSRKRTVLTRESEAVREADARTGRTWIISNPESKNRLNEPVGYKLHAHNQPTLLADPGSSIARRAAFATKDLWVTRYADDERYPTGDFVNQHSGGAGLPSYIAQDRDIDGQDIVVWHTFGLTHFPRVEDWPIMPVDTVGFKLRPEGFFDRSPVLDVPANPSQSGSHCHG

SEQ ID NO: 21 - protein, 650 aa, Arabidopsis thaliana
MNTSILAILFLIQCVFTLGLHFHPLDPLTPQEINKTSFIVKKSHLGMLKDLTFHYLDLEEPNKSHVLQWLSPNPSKKPPPPRRRSFVVVRAGGQTYELIIDLTTSKIASSRIYTGHGFPSFTFIELFKASKLPLTYPPFKKSILDRSLNISEVSCIPFTVGWYGETTTRRELKASCFYRDGSVNVFTRPIEGITVTIDVDSMQVIKYSDRFRKPIPDKEGNDFRTKHRPFPFFCNVSDTGFKILGNRVKWANWKFEVGFTARAGVTISTASVLDPRTKRFRRVMYRGHVSETFVPYMDPTYEWYYRTFMDIGEFGFGRSAVNLQPLIDCPQNAAFLDGHVAGPDGTAQKMTNVMCVFEKNGYGASFRHTEINVPGQVITSGEAEISLVVRMVATLGNYEYIVDWEFKKNGAIRVGVDLTGVLEVKATSYTSNDQITENVYGTLVAKNTIAVNHDHYLTYYLDLDVDGNGNSLVKAKLKTVRVTEVNKTSSRRKSYWTVVKETAKTEADGRVRLGSDPVELLIVNPNKKTKIGNTVGYRLIPEHLQATSLLTDDDYPELRAGYTKYPVWVTAYDRSERKAGGFYSDRSRGDDGLAVWSSRNREIENKDIVMWYNVGFHHIPYQEDFPVMPTLHGGFTLRPSNFFDNDPLIG

SEQ ID NO: 22 - protein, 655 aa, Hansenula polymorpha
PARPAHPLDPLSTAEIKAATNTVKSYFAGKKISFNTVTLREPARKAYIQWKEQGGPLPPRLAYYVILEAGKPGVKEGLVDLASLSVIETRALETVQPILTVEDLCSTEEVIRNDPAVIEQCVLSGIPANEMHKVYCDPWTIGYDERWGTGKRLQQALVYYRSDEDDSQYSHPLDFCPIVDTEEKKVIFIDIPNRRRKVSKHKHANFYPKHMIEKVGAMRPEAPPINVTQPEGVSFKMTGNVMEWSNFKFHIGFNYREGIVLSDVSYNDHGNVRPIFHRISLSEMIVPYGSPEFPHQRKHALDIGEYGAGYMTNPLSLGCDCKGVIHYLDAHFSDRAGDPITVKNAVCIHEEDDGLLFKHSDFRDNFATSLVTRATKLVVSQIFTAANYEYCLYWVFMQDGAIRLDIRLTGILNTYILGDDEEAGPWGTRVYPNVNAHNHQHLFSLRIDPRIDGDGNSAAACDAKSSPYPLGSPENMYGNAFYSEKTTFKTVKDSLTNYESATGRSWDIFNPNKVNPYSGKPPSYKLVSTQCPPLLAKEGSLVAKRAPWASHSVNVVPYKDNRLYPSGDHVPQWSGDGVRGMREWIGDGSENIDNTDILFFHTFGITHFPAPEDFPLMPAEPITLMLRPRHFFTENPGLDIQPSYAMTTSEAKRAV

(19) United States
(12) Patent Application Publication
(10) Pub. No.: US 2009/0055964 A1
(43) Pub. Date: Feb. 26, 2009

(54) MPO1 GENE AND PROTEIN AND METHODS OF USE

(76) Inventors: John G. Gelesko, Blacksburg, VA (US); William G. Heim, Gettysburg, PA (US); George Heim, Gettysburg, PA (US)

Correspondence Address: LATIMER, MAYBERRY & MATTHEWS IP LAW, LLP, 13873 PARK CENTER ROAD, SUITE 106, HERNDON, VA, 20171, US

(21) Appl. No.: 11/775,802

(22) Filed: Jul. 10, 2007

Related U.S. Application Data
(60) Provisional application No. 60/807,035, filed on Jul. 11, 2006.

(51) Int. Cl. A01H 1/00; C07H 21/04; C12N 15/63; C12N 5/10; C12N 9/14; C12Q 1/34

(52) U.S. Cl. 800/278; 536/23.2; 435/320.1; 435/410; 435/195; 435/18

(57) ABSTRACT

The present invention provides genes, proteins, and cells comprising recombinant methylputrescine oxidase (MPO) from Nicotiana tabacum. The gene and protein may be used to create transgenic plants having altered nicotine and/or alkaloid production, or can be used to identify compounds that affect nicotine and alkaloid production, in plants.

### BACKGROUND OF THE INVENTION

1. Field of the Invention

The present invention relates to the field of molecular biology. More particularly, the present invention relates to the cloning and expression of an MPO1 gene from Nicotiana tabacum, and use of the encoded protein in research, plant engineering, and medicinal and consumer products fields.

2. Description of Related Art

Nicotine is synthesized in the roots of many Nicotiana species. Nicotine is subsequently transported from the roots to the shoots where it serves as a natural insecticide, because it binds and hyperstimulates insect acetylcholine receptors, resulting in paralysis of the insect. It also has an effect on animal physiology, acting on the central nervous system. It is well known as the active ingredient in tobacco products, and its effects on the central nervous system are recognized as key in the addictive qualities of tobacco products.

Nicotine is comprised of a pyridine ring and an N-methylpyrrolidine ring. An essential step in pyridine ring biosynthesis is the decarboxylation of quinolinic acid to form nicotinic acid, a reaction carried out by quinolinate phosphoribosyl transferase (QPT). The N-methylpyrrolidine ring of nicotine is formed by the spontaneous cyclization of N-methylaminobutanal generated by the oxidative deamination of N-methylputrescine by a copper-containing N-methylputrescine oxidase (MPO) activity. Nicotine biosynthesis is also under genetic control by the A and B loci in Nicotiana tabacum. Expression of all known nicotine biosynthetic genes and MPO enzyme levels are reduced in tobacco roots with the mutant aabb genotype.

Production of nicotine shares some similarities with production of alkaloids, including those produced by coca plants and other similar plants. Many of these alkaloids are known for their effects on the central nervous system, and their use as or in drugs (both legal and illegal) is well documented. Production of nicotine and these alkaloids are linked through the process of production of N-methyl-pyrrolinium and its salt. This compound serves as the precursor for the N-methylpyrrolidine ring of both nicotine and the alkaloids.

The economic effects of use, addiction, and abuse of nicotine and alkaloid drugs is well known throughout the world. While these compounds provide exceptional sources of revenue for tobacco companies, pharmaceutical companies, and drug traffickers, their effects on human health and behavior are of concern to many in the health care field. Their effects on the central nervous system are also of concern to governmental agencies tasked with protection of the health and safety of citizens. While various approaches to regulating and controlling production and use of products containing nicotine and other alkaloids have been taken, there still exists a need for improved methods of regulating production and use of these compounds. Furthermore, related compounds, such as compounds of the tropane alkaloid class, find beneficial use in or as pharmaceuticals. For example, some of the tropane alkaloids are used as mydriatics, antispasmodics, and antidotes to fertilizer or nerve-gas poisonings. There is thus a desire in the pharmaceutical and medical fields for improved production of these compounds for treatment of subjects who can benefit from their activities.

As has been established in recent years, transgenic tobacco can be used as a production system for expressing pharmaceuticals and industrial proteins. Beneficial characteristics of the system include, but are not limited to, ease of transformation of exogenous genes, generation to generation stability, low risk of gene flow in the environment via pollen compared to other crop plants, ability to express high levels of protein per unit of biomass, and competitive development time compared to other cell-based systems. These other systems, such as mammalian cells, bacterial systems, and yeast fermentation tanks, require highly sophisticated factories and cost millions of dollars to build and maintain. The tobacco production system can be scaled up or down easily and cost-effectively because it requires readily available farmland instead of production facilities for growth of the transgenic cells. Transgenic tobacco has been used to produce different kinds of human pharmaceuticals, notably antibodies such as full-length immunoglobulin G antibody (Hiatt et al., Nature 342:76-78), multimeric secretory antibody (Ma et al., Science 268:716-719), and many functional antibody fragments. A wide range of other kinds of transgenic proteins that can be used for human health have also been expressed, such as lysosomal storage proteins (U.S. Pat. No. 5,929,304) and a severe acute respiratory syndrome (SARS) S protein that can potentially be used as a vaccine (Pogrebnyak et al., Proc Natl Acad Sci USA 102(25):9062-7). Transgenic plants that have reduced or no levels of nicotine would be desirable in a production system for high-value pharmaceutical or medicinal compounds.

The current state of the art discloses some uses for the MPO gene related to transgenic tobacco. For example, Conkling (U.S. Patent Application Publication No. 2006/0060211) claims a tobacco product with reduced levels of MPO enzyme, among other enzymes. However, this publication does not disclose cloning or a sequence for the MPO gene, or its gene or protein sequence. Albino et al. (U.S. Patent Application Publication No. 2006/0157072) discloses a method of reducing nicotine consumption of a tobacco user by providing genetically modified tobacco comprising an exogenous fragment of a gene, such as the MPO gene. However, the sequence of the MPO gene, or a fragment thereof, is not provided.

There is a need in the art for cloning and sequencing of the MPO gene so that it can be used in transgenic plants to modulate production of high-value pharmaceutical compounds, as well as offer an alternative as a low-nicotine background for transgenic tobacco production systems.

### SUMMARY OF THE INVENTION

The present invention addresses needs in the art by providing proteins, genes, compositions, and methods for the regulation and production of nicotine and certain compounds of the alkaloid class. As used herein, unless otherwise specifically noted, the term “alkaloid” is used to refer to the following compounds and chemically related compounds: nicotine, pyridine alkaloids, pyrrolidine alkaloids, and tropane alkaloids. The present invention provides, for the first time, a copper-containing diamine oxidase that is involved in the biosynthesis of a variety of alkaloid compounds derived from either N-methylpyrrolinium or pyrrolinium salts. The oxidase, named MPO1 for N-methylpyrrolinium oxidase, converts N-methyl-putrescine to N-methyl-pyrrolinium salt, which is ultimately incorporated into either nicotine or various alkaloids. The invention provides the ability to regulate nicotine and alkaloid production in various plants by introducing the MPO1 gene and/or protein into cells, such as cells of a plant, by supplementing existing levels of MPO1 protein in a cell that already produces it, or by introducing a defective MPO1 gene, such as a non-functional deletion mutation, into a cell that normally produces the MPO1 protein. Products derived from the cells and organisms engineered by the invention can benefit the medical industry by providing central nervous system affecting compounds, such as nicotine and the various alkaloid compounds made by plants. The products can also provide a low-nicotine background for transgenic plant production systems. Furthermore, the gene and protein can be used to identify compounds that affect their activities.

In a first aspect, the invention provides nucleic acids comprising the MPO1 coding region and/or gene from Nicotiana tabacum, and all nucleotide sequences encoding the N. tabacum protein encoded by the MPO1 gene. It likewise provides for all changes to the MPO1 gene that do not significantly affect its encoded MPO1 protein. In an exemplary embodiment, the invention provides nucleic acids comprising the sequence of SEQ ID NO:1. The invention further provides compositions and cells comprising nucleic acids of the invention. Then again, the MPO1 gene may be used for identifying naturally occurring or random mutation-induced variant MPO1 alleles with desired properties.

In another aspect, the invention provides peptides, polypeptides, and proteins encoded by portions or all of the nucleic acids of the invention. The peptide, polypeptides, and proteins comprise part or all of an MPO1 protein and can be used as enzymes, to raise antibodies, and to identify inhibitors or activators of the protein. Compositions and cells comprising the peptides, polypeptides, and proteins are also provided.

In an additional aspect, methods of making an MPO1 protein, or a portion thereof, are provided. In general, in some embodiments, the method comprises providing an MPO1-encoding nucleic acid and expressing the MPO1 from that nucleic acid. In other embodiments, the method comprises partially or totally chemically synthesizing the MPO1 protein from smaller subunits, such as from individual amino acids or from peptides. The method can include making modifications to the primary amino acid sequence during or after synthesis of the MPO1 protein, for example by introducing copper or another metal into the protein, or modifying one or more residues by addition of a chemical moiety, such as by glycosylation or the like. The method of this aspect of the invention may also comprise isolating or purifying the MPO1 protein, at least to some extent.

As mentioned above, the invention provides recombinant cells comprising the MPO1 gene and/or protein, or portions of these. Thus, the invention provides recombinant, such as transgenic, cells comprising the MPO1 protein or portions thereof. For example, the invention provides transgenic plants that express a recombinant MPO1 protein from a heterologous MPO1 gene (i.e., an MPO1 gene that is not naturally present in the recombinant cell). Alternatively, the invention provides transgenic plants in which one or more copies of an MPO1 gene is present, in addition to a heterologous MPO1 gene present in the cell normally.

In yet a further aspect, the invention provides a method of altering the production of nicotine and/or one or more alkaloid compounds in a plant cell. The method generally comprises introducing into a cell at least one copy of an MPO1-encoding gene or a gene that encodes a non-functional MPO1 protein, and expressing the encoded protein. Where the gene encodes a functional protein, production of nicotine and/or alkaloids is increased as a result of increased conversion of precursor compounds within the relevant biosynthetic pathway, and in particular N-methyl-pyrrolinium salt. Where the gene encodes a non-functional protein, production of nicotine and/or alkaloids is decreased as a result of decreased conversion of precursor compounds within the relevant biosynthetic pathway. In addition, where the coding region is operably linked to one or more regulable control elements, the expression of the gene can be regulated by cellular signals, which can be naturally induced or induced through manipulation of the cell's environment (e.g., by exposing the cell to one or more chemical compounds).

In yet another aspect, the invention provides a method of identifying substances that affect the activity of an MPO1 protein. In general, the method comprises providing an MPO1 protein, or a polypeptide comprising MPO1 activity, exposing the protein or polypeptide to one or more substances, and determining the activity of the MPO1 protein or polypeptide. In embodiments, the method further comprises comparing the determined activity to a known activity for the protein or polypeptide in the absence of the substance(s) to determine if the activity has changed. The alteration may be an increase in activity or a decrease in activity. The method may be used to screen individual substances or many substances at once. For example, the method may be a high-throughput screening (HTS) method.

### BRIEF DESCRIPTION OF THE DRAWINGS

The accompanying drawings, which are incorporated in and constitute a part of this specification, illustrate various features and details of embodiments of the invention, and together with the written description, serve to explain certain principles of the invention.

FIG. 1 is a schematic diagram of nicotine synthesis and early steps in alkaloid synthesis in plants.

FIG. 2A is a Northern blot showing that MPO-like transcript levels in mutant LA21 roots were lower than those in B21 roots.

FIG. 2B is a bar graph showing that transcript levels were significantly reduced in mutant LA21 roots during control and MJA treatments.

FIG. 2C shows a DNA blot analysis of B21 genomic DNA, indicating the presence of a small multigene family of approximately six members.

FIG. 3 shows a comparison of the MPO protein sequence with other copper-containing proteins.

FIG. 4A is a protein gel showing enrichment of the NtMPO1 from cell lysates.

FIG. 4B is a bar graph showing that the TRX-NtMPO1 extracts showed substrate-specific amine oxidase activity over background levels.

### DETAILED DESCRIPTION OF VARIOUS EMBODIMENTS OF THE INVENTION

Reference will now be made in detail to various exemplary embodiments of the invention, examples of which are illustrated in the accompanying drawings. The following detailed description is provided to give the reader a better understanding of certain details of aspects of the invention, and should not be understood as a limitation of the invention.

In one aspect, the invention provides nucleic acids comprising the MPO1 coding region and/or gene from Nicotiana tabacum, and all nucleotide sequences encoding the N. tabacum protein encoded by the MPO1 gene. It likewise provides for all changes to the gene that do not significantly affect its encoded MPO1 protein, for example by significantly changing its enzymatic activity. Those of skill in the art are well aware of numerous suitable changes that can be made according to the invention. For example, any change to the nucleic acid sequence of an MPO1 gene that does not result in a change in the primary amino acid sequence of the encoded wild-type protein are encompassed. Likewise, any change in the nucleotide sequence that results in a conserved change in the amino acid sequence of the encoded protein is encompassed. Various residues that are preferably not altered are disclosed below in the context of the protein sequences. Corresponding changes to the nucleic acid sequences are accordingly taught. The invention encompasses the use of the MPO1 coding region and/or gene to alter MPO1 expression.

In general, a nucleic acid of the invention comprises at least 50% sequence identity to an MPO1 gene, such as SEQ ID NO:1. For example, it may comprise at least 60%, at least 70%, at least 80%, at least 90%, at least 95%, at least 98%, at least 99%, or about 100% identity. Of course, and specific number within these ranges may be used without the need to disclose each particular number individually herein. Percentages of identity may also be described with reference to any sub-sequence of an MPO1 gene, such as any sub-sequence of SEQ ID NO:1. Unless otherwise indicated, the length of the sub-sequence is not critical, and any length, covering any range of nucleotides is envisioned by the invention, without the need to particularly identify each range by nucleotide number.

In an exemplary embodiment, the invention provides nucleic acids comprising the sequence of SEQ ID NO:1. This sequence is the sequence of the wild-type M. tabacum MPO1 gene, and serves as an exemplary sequence of the invention. Numerous alterations to the sequence, including deletions, additions, and substitutions may be made without significantly changing the enzymatic activity or other properties (e.g., antigenicity) of the encoded protein.

According to the invention, all nucleic acids comprising the nucleotide sequence of SEQ ID NO: 1 are provided. Thus, the invention provides probes and primers comprising a portion of the sequence of SEQ ID NO:1, or a sequence with sufficiently high identity to SEQ ID NO:1 to specifically bind to it under chosen conditions. It is to be noted at this point that reference to a nucleic acid sequence, unless otherwise specifically noted, includes a reference to the complementary sequence as well. Numerous computer programs are available for design of probes and primers with various levels of identity to a target sequence for binding under various hybridization conditions. Probes and primers may be of any suitable length, but are typically 10-30 nucleotides in length. Fragments of an MPO1 gene may also be provided, where the fragment comprises any number of nucleotides that are fewer than the corresponding wild-type sequence. One of skill in the art can immediately understand the various different lengths of fragments described herein without the need for each particular length of fragment to be specifically listed.

Exemplary nucleic acids of the invention include vectors, such as plasmids, phages, phagemids, plant viruses, and the like. Vectors comprise the coding region of an MPO1 gene of the invention, or a portion thereof, along with other sequences, such as sequences for expression of the gene or portion thereof, sequences for integration of the nucleic acid into a cell, and particularly into the host genome, or for maintenance of the nucleic acid in a host cell in an autonomous state. Vectors may also comprise any of the other sequences, cassettes, etc. that are typically found in vectors for manipulation, expression, maintenance, etc. of nucleic acids in host cells. For example, they may comprise promoters or other elements that control expression of mRNA from genes, including, but not limited to, upstream regulatory elements, terminators, protein initiation sequences, and the like. Sequences on the vector may function independently of other sequences or may be operably linked, such as by way of a promoter controlling expression of a coding region. Sequences that are operably linked to others need not be the sequences that are naturally found linked to the other sequences. For example, a promoter from one organism (e.g., the host organism) may be operably linked to an MPO1 gene to drive expression in the host organism, preferably in some sort of controlled way (e.g., tissue specific, developmental specific, etc.).

Any of the nucleic acids of the invention can comprise part of a composition. As a general matter, a composition according to this aspect of the invention comprises a nucleic acid of the invention and at least one other substance. Compositions thus may comprise many nucleic acids and one or more other substances. For example, the nucleic acids may comprise part of an in vitro amplification reaction or an in vitro protein expression reaction. They also may comprise part of a transformation reaction, such as a composition comprising intact cells. They further may comprise part of a cell lysate. Many other exemplary compositions can be envisioned, and all such compositions need not be described in detail here for those of skill in the art to recognize them.

The invention provides host cells comprising an exogenously-supplied MPO1 gene, or a portion thereof. Such host cells are also referred to herein as recombinant cells to more accurately reflect that they may be cells that were transformed, transfected, etc., or cells derived from such cells, such as cells of a lineage from an original transformant. Transgenic cells, individually or as part of a transgenic multicellular organism, are also encompassed by the terms host cell and recombinant cell. The type of cell is unlimited, including both prokaryotic cells and eukaryotic cells.

In addition to the above uses, the invention includes using MPO1 to find naturally occurring or mutation-induced variants. For example, the MPO1 gene or mRNA transcribed from this gene can be used as a probe to look for other variants of the gene by nucleic acid hybridization methods, such as DNA microarrays or PCR amplification. Once a desired variant is found, it can be used in a variety of ways, such as the MPO1 protein variant being used to generate antibodies, and the like. The invention also includes marker-assisted breeding for MPO1 alleles with desired properties. Thus, the invention is not limited to use of the nucleic acids to create transgenic plants.

In another aspect, the invention provides peptides, polypeptides, and proteins encoded by portions or all of the nucleic acids of the invention. Thus, the invention provides MPO1 proteins. It likewise provides polypeptides having MPO1 enzymatic activity. It further provides peptides comprising MPO1 amino acid sequences, which may have any number of activities or properties. For example, MPO1 peptides may comprise MPO1 activity (i.e., the ability to convert N-methyl-putrescine to N-methyl-pyrrolinium salt in vitro or in vivo), may comprise diamine oxidase activity with other primary or secondary diamine substrates (for example, 1,5-diamine pentane leading to the biosynthesis of anabasine and related alkaloids), may comprise antigenic portions of an MPO1 protein (which can be used to develop antibodies that specifically bind to an MPO1 protein), or may provide inhibitory activities against other MPO1 proteins, for example by acting as competitors for MPO1 substrates. As used herein, a peptide is a poly-amino acid molecule comprising two to about twenty amino acids covalently linked to form a chain; a polypeptide is a poly-amino acid molecule comprising about twenty to one hundred or more amino acids covalently linked to form a chain; whereas a protein comprises about twenty to one hundred or more amino acids covalently linked to form a chain, in which the complete sequence, when folded properly into a three-dimensional structure is recognized as a functional unit within the context of a living cell, such as to provide an enzymatic activity or a structural component of the cell. Thus, a protein can be a polypeptide, where the polypeptide has a recognized activity within a cell. For the purposes of this document, polypeptide and protein are used interchangeably when in reference to functional units with the context of cells. Thus, an MPO1 polypeptide may comprise MPO1 protein activity, yet not comprise the full or wild-type MPO1 sequence. It likewise may comprise little or no detectable MPO1 activity, but have clear sequence identity to a wild-type MPO1 protein.

Accordingly, the peptides, polypeptides, and proteins of the invention comprise part or all of an MPO1 protein. The MPO1 protein may be any MPO1 protein, which has the activity of converting N-methyl-putrescine to N-methyl-pyrrolinium salt in vitro or in vivo, and has high sequence identity to the MPO1 protein of SEQ ID NO:2, at least over a portion of the peptide, polypeptide, or protein sequence of interest. For example, it may comprise the amino acid sequence of SEQ ID NO:2. Alternatively, it may comprise a sequence taken from SEQ ID NO:2, which is 10 or more amino acids in length, such as 10-20 amino acids in length, 15-25 amino acids in length, 20-40 amino acids in length, 25-50 amino acids in length, or 30 or more amino acids in length. It is important to note that, as used herein, each value expressed (whether the value be in reference to a protein, nucleic acid, activity, or any other concept) inherently includes a range of values about the recited value, where the inherent range is 10%. Thus, a peptide that is disclosed as being 20 residues in length is to be understood as having anywhere from 18 to 22 residues. Where a value would require a fraction of a unit of measure that cannot exist (e.g., a fraction of an amino acid), it is to be understood that the range is to be rounded up or down to achieve the closest whole number. Thus, for example, where a peptide is disclosed as having 21 amino acids, the range of 18.9 to 23.1 amino acids would be reduced to 19 to 23 amino acids in order to achieve whole numbers.

In preferred embodiments, the sequence of the protein, polypeptide, or peptide comprises the sequence of SEQ ID NO:2, or a portion thereof. Of course, due to the conserved nature of many amino acids, the proteins, polypeptides, and peptides of the invention also encompass those molecules that show high primary sequence identity to SEQ ID NO:2 or portions of it. For example, one or more conservative changes may be made in the primary sequence of SEQ ID NO:2 or portions of it to achieve proteins, polypeptides, and peptides according to the invention, which have the same activity as the corresponding wild-type sequence (although not necessarily the same level of activity). Alterations to SEQ ID NO:2 may result in proteins, polypeptides, and peptides having less than 50% amino acid identity to SEQ ID NO:2 without significantly affecting the activity of interest. For example, polypeptides having from 50%-99%, or any particular percent within this range, sequence identity to the MPO1 of SEQ ID NO:2 may be created or discovered, which are equivalent in structure and function to the protein comprising SEQ ID NO:2. As used herein, comparisons of sequences (be they amino acid or nucleic acid) can be performed using any number of publicly available computer programs or by manual comparison of the sequences of interest. Where a comparison is performed, the percent identity is expressed in terms of the smallest or shortest of the sequences compared. Percent identity does not include any consideration of similarity.

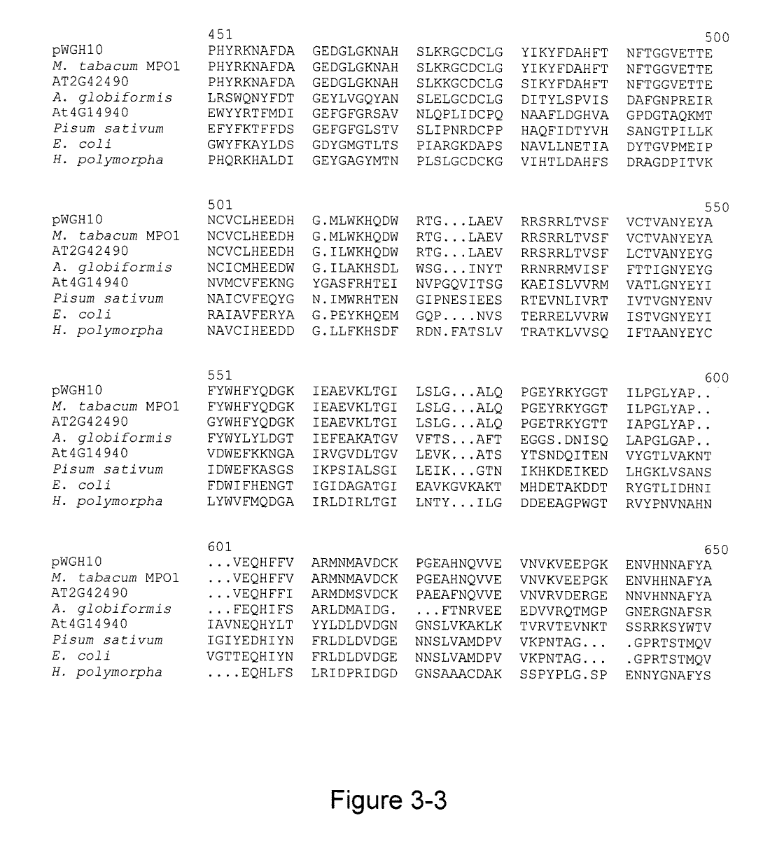

The MPO1 sequence of SEQ ID NO:2 is the first sequence of a copper-containing methylputrescine oxidase disclosed. Sequence comparisons to known sequences in databases show it to be a member of the class of proteins known as amine oxidases. All members of this class contain conserved residues and regions, which are implicated in structural and enzymatic functions of the class members. For example, as shown in FIG. 3, all class members have conserved residues at regions spanning residues 490-510, 540-555, and 590-615. In particular, all class members have a conserved asparagine at the position corresponding to residue 495 of SEQ ID NO:2, a conserved tyrosine at position 496, and a conserved negative residue (aspartic acid or glutamic acid) at position 497. They also have conserved histidine residues at the position corresponding to residues 546 and 548 of SEQ ID NO:2. They further have a conserved histidine at the position corresponding to residue 712 of SEQ ID NO:2. These residues, and in particular the conserved histidines, are thought to be involved in copper chelation in the properly folded proteins. The conserved asparagines, tyrosines, and glutamic or aspartic acids could participate in the active site of the protein. In addition to these residues, residues that may be involved in gross folding of the protein into its proper three dimensional structure, such as proline residues found at certain positions throughout SEQ ID NO:2, may be conserved, and thus avoided as a site of alteration within the protein. Thus, those of skill in the art, while being able to make changes to the sequence of SEQ ID NO:2 to create other proteins according to the present invention, will recognize that these conserved residues should not be altered if the activity of the protein is to be maintained. Alternatively, if the goal is to abolish or reduce enzymatic activity, one would target these residues. Of course, numerous methods are known for making changes to amino acid sequences, the simplest now being mutagenesis of the underlying nucleic acid.

The peptides, polypeptides, and proteins of the invention can be used as enzymes or pseudo-enzymes, to raise antibodies, and to identify inhibitors or activators of the protein, among other things. While the full-length protein of SEQ ID NO:2 is an exemplary protein having MPO1 enzymatic activity, it is not the only sequence capable of providing this activity. Thus, portions of SEQ ID NO:2, such as N-terminal or C-terminal truncations, may provide equivalent activity. Likewise, proteins having additional amino acids at one or both ends and/or within the interior of SEQ ID NO:2 may provide equivalent activity. The proteins of the invention may be used in numerous applications, both in vitro and in vivo, and in particular to provide the enzymatic activity of the MPO1 wild-type protein. Proteins, polypeptides, and peptides of the invention can also be used as antigenic molecules to generate antibodies that are specific for the MPO1 protein. Antibodies raised against these molecules can be used to detect the proteins in cells, in affinity purification protocols, and to probe for structurally related molecules in various organisms.

The invention provides for all compositions comprising the peptides, polypeptides, and proteins of the invention. In general, a composition comprises at least one molecule of a peptide, polypeptide, or protein of the invention and one other substance. As with compositions comprising the nucleic acids of the invention, the other substance is not limited in structure or amount, and thus can be a solvent (e.g., water), a salt, another peptide, polypeptide, or protein, or a complex mixture of simple and complex substances and compounds, such as would be found in a cell lysate. In embodiments, the composition comprises one or more purified (to any extent) peptides, polypeptides, or proteins of the invention. When referring to a peptide, polypeptide, or protein within the context of a composition, it is to be understood that the peptide, polypeptide, or protein may be present in one or multiple copies within the composition, and that all numbers of copies are included when a reference to “a protein” etc. is made. In embodiments, the composition comprises, in addition to a protein of the invention, at least one substrate for the protein and necessary or suitable other substances for performing an assay for the activity of the protein. In embodiments, one or more inhibitors or activators of the protein is present in the composition. Thus, the composition may be a composition for assaying activity of the protein, polypeptide, or peptide of the invention.

The invention further provides cells comprising the peptides, polypeptides, and proteins of the invention. Typically, the cells are recombinant cells comprising a peptide, polypeptide, and/or protein of the invention that is either not naturally present in the cell or is present at a particular amount or level due to expression of the protein at a level that is different (higher or lower) than is normally seen in the cell in nature. For example, the cells may be recombinant plant cells expressing MPO1 (or a portion thereof) from an exogenously provided nucleic acid. The MPO1 may be from the same plant, but exogenously provided, and may be expressed in cells that normally express the MPO1 protein or in cells that do not normally express the protein. Likewise, the cell may be a cell other than a plant cell, such as one that does not normally express the MPO1 protein (or portions thereof).

Thus, the invention provides recombinant, such as transgenic, cells comprising the MPO1 protein or portions thereof. For example, the invention provides transgenic plants that express a recombinant MPO1 protein from a heterologous MPO1 gene (i. e., an MPO1 gene that is not naturally present in the recombinant cell). Alternatively, the invention provides transgenic plants in which one or more copies of an MPO1 gene is present, in addition to a heterologous MPO1 gene present in the cell normally. Then again, the invention also provides transgenic plants in which an endogenous MPO1 gene is replaced by a non-functional MPO1 gene, for example a gene with a deletion rendering the encoded protein non-functional. In such a case, a cell that normally would produce an MPO1 protein does not, and the biochemical pathway involving MPO1 is shut down or significantly reduced. Accordingly, knock-out cells and transgenic plants are provided. The invention further provides for the use of Viral Induced Gene Silencing (VIGS) and RNA Interference(RNAi) as mechanisms of decreasing MPO1 expression levels.

Transgenic plants comprising an exogenous MPO1 gene are provided by the invention. For example, tobacco plants can be transformed with an exogenous MPO1 gene using methods known in the art, such as Agrobacterium tumefaciens-mediated transformation, viral mediated transformation, or particle bombardment to produce a transgenic plant with a change in MPO1 expression. Any type of plant can be employed for this invention that is capable of being transformed with an foreign gene. In one embodiment, the plant is a tobacco plant, such as Burley, Oriental, or Flue-cured. Plants that already have modified levels of alkaloid production, such as plants with naturally low levels of nicotine or plants that have been previously modified with another exogenous gene, are covered by this invention, as long as the introduction of the MPO1 gene into the plant results in further modifications of MPO1 expression and/or alkaloid production. As another example, the MPO1 gene can be incorporated into the plastids of the plant, which has the potential to result in high transgene expression levels as well as has the absence of gene silencing and position effect variations. However, transformation of the plastids usually results in sequestration of foreign proteins in the organelle which may or may not be beneficial. In general, the transgenic plant is produced by exposing at least one plant cell of a selected variety to an exogenous DNA construct having regulatory sequences including a promoter operable in a plant cell and DNA containing at least a portion of a DNA sequence encoding for MPO1. The promoter can be tissue specific, where expression of the MPO1 gene is greatest in the roots, stems, and/or leaves of the plant. The promoter can be constitutive or inducible, such as a stress response, light response, or chemically inducible promoter. The DNA is operably associated with the promoter, the plant cell is transformed with the DNA construct, the transformed cells are selected, and at least one transgenic tobacco plant is regenerated from the transformed cells. Transformation of the transgenic gene can occur in a non-homologous (illegitimate) or homologous recombination event, although homologous events are infrequent (usually 10⁻⁴ to 10⁻⁵ homologous versus non-homologous events). The transgenic plants produced can result in a change of expression of the MPO1 gene, and subsequently, a change in the amount of nicotine and/or alkaloids, such as tropane alkaloids, produced by the transgenic plant.

In some embodiments of the invention, the transgenic plant has increased levels of MPO1 expression, such as increased levels of MPO1-specific mRNA and/or higher levels of MPO1 protein, which result in increased levels of nicotine and/or alkaloids in the plant. Transgenic tobacco with these characteristics can be used to produce more potent tobacco products that have increased levels of nicotine but lower levels of other toxic chemicals, such as tar, benzene, hydrogen cyanide, pesticides, etc. Tobacco products include, but are not limited to, smoking materials such as cigarettes, cigars, pipe tobacco, snuff, chewing tobacco, gum, and lozenges. Transgenic tobacco with higher levels of nicotine would also be beneficial for the production of tobacco products used in tobacco cessation kits and programs because not as many plants would need to be grown and harvested. Such kits and programs are also embodiments of the invention.

In other embodiments, the transgenic plant has decreased levels of MPO1 expression, such as decreased levels of MPO1-specific mRNA and/or lower levels of MPO1 protein, which result in decreased levels of nicotine and/or alkaloids in the plant. Reduction of MPO1 expression can occur in any way that reduces the levels of MPO1 mRNA and/or protein, such as use of antisense RNA, RNA interference, gene targeting, naturally occurring or induced deletions or point mutations, and the like. Transgenic tobacco with decreased levels of alkaloids can be utilized to make low nicotine tobacco products. As another example, tobacco plants with lower or no nicotine levels would be preferable for production of transgenic proteins, such as human pharmaceutical drugs, because the transgenic protein would not need to be purified away from as much or any of the nicotine in the plant.

In addition to nicotine, other high-value alkaloids are found in the same pathway as MPO1, including, but not limited to, hygrine, pyridine, nortropane, and tropane-type alkaloids. In an embodiment, the transgenic plant has increased levels of MPO1 expression, which results in increased levels of high-value alkaloids, such as cocaine or scopolamine (used in transdermal patches to combat motion sickness or as antidotes to nerve gas agents used during unconventional warfare). High-value alkaloids, although widely misused in some cases, can also be beneficial for medicinal purposes. A transgenic plant can overexpress MPO1 or have changed expression levels of MPO1 regulatory genes that result in increased alkaloid levels in plant species that already produce them. Alternatively, a plant that does not currently produce these compounds can become producers of this class of high-value alkaloids, by the addition of a MPO1 gene as well as other genes in the tropane-alkaloid pathway.

The cells of the invention may be any type of cell, either prokaryotic or eukaryotic. Thus, the cells may be bacterial cells, fungal cells, mammalian cells, or plant cells. They may be individual, free-living cells, such as bacterial cells in culture, or may be part of a multi-cellular organism, such as plant cells of a living plant. Accordingly, examples of cells include, but are not limited to, bacterial cells such as Escherichia coli, yeast cells such as Saccharomyces cerevisiae, and plant cells, such as those belonging to the family of Solanaceae (including but not limited to members of the genera Atropa, Hyoscamus, Mandrogora, Nicotiana, Lycopersicon, Solanum, Scopolia, and Capsicum), or the genera of Erythroxylum. In one embodiment, the cells are found as hairy root cultures that have been transformed with an exogenous gene using Agrobacterium rhizogenes.

In another aspect, methods of making an MPO1 protein, or a portion thereof, are provided. In general, there are two main ways of performing the method. The first way, which is most applicable to longer molecules, such as polypeptides and proteins, is to express the molecules from a nucleic acid encoding them. In these embodiments, the method generally comprises providing an MPO1-encoding nucleic acid or a portion thereof and expressing the MPO1 or a portion thereof from that nucleic acid. The second way of practicing the method is most applicable to shorter molecules, such as peptides and short polypeptides. In these embodiments, the method generally comprises partially or totally chemically synthesizing the MPO1 protein from smaller subunits, such as from individual amino acids or from peptides. Regardless of the embodiment chosen, the method can include making modifications to the primary amino acid sequence during or after synthesis of the MPO1 protein, polypeptide, or peptide, for example by introducing copper or another metal into the amino acid chain, or modifying one or more residues by addition of a chemical moiety, such as by glycosylation or the like. The method of this aspect of the invention may also comprise isolating or purifying the resulting protein, polypeptide, or peptide, at least to some extent. Methods of purifying peptides, polypeptides, and proteins are well known in the art and thus need not be detailed herein. For example, purification may be achieve by one or more column chromatography steps based on charge (e.g., anion exchange, cation exchange), size, and/or affinity, or using preferential precipitation, and the like. In embodiments, the protein, polypeptide, or peptide is pure to the extent that contaminants are undetectable using normal detection methods. In other embodiments, the protein, polypeptide, or peptide is partially pure in that at least one substance that is normally found in the same natural environment as the protein, polypeptide, or peptide, is not present in the environment in which the protein, polypeptide, or peptide is present. In some embodiments, the method further comprises assaying the protein, polypeptide, or peptide for one or more characteristics (e.g., enzymatic activity, size, antigenicity). Thus, the invention encompasses the use of all or a portion of the MPO1-encoding nucleic acid to make a MPO1-protein, or portions thereof.

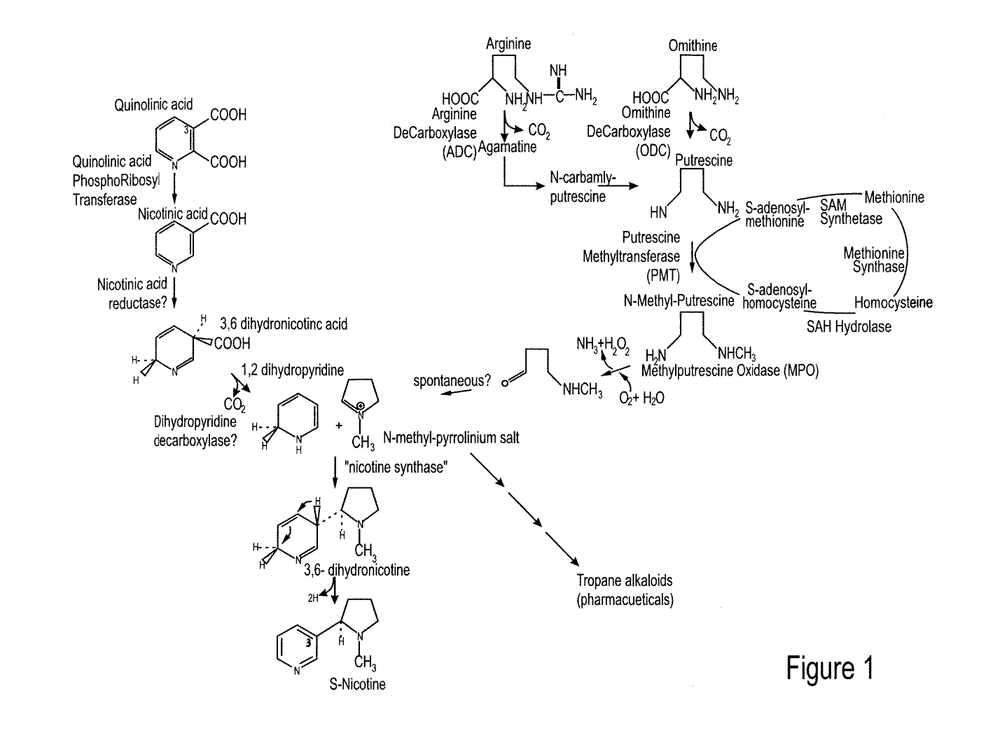

In yet a further aspect, the invention provides a method of altering the production of nicotine and/or one or more alkaloid compounds in a plant cell. The MPO1 protein of the invention, exemplified by SEQ ID NO:2, is involved in the conversion of N-methyl-putrescine to N-methyl-pyrrolinium in tobacco plants. As can be seen from FIG. 1, the MPO1 enzyme is involved in production of a compound that is necessary for ultimate production of both nicotine and the various other alkaloid compounds produced by plants. Through the discovery, isolation, and characterization of this protein and its underlying gene, the present invention provides, for the first time, the ability to regulate, at a genetic and protein level, the production of nicotine and other alkaloid compounds in cells, and in particular in plant cells. More specifically, by introducing into cells an exogenous nucleic acid encoding MPO1 or a portion of it, production of nicotine and/or alkaloids via the pathway depicted in FIG. 1 can be increased (where a functional MPO1 is supplied) or decreased (where a non-functional MPO1 is supplied as a replacement or inhibitor for the endogenous MPO1). As discussed above, transgenic plants comprising either functional MPO1 or non-functional MPO1 may be created to regulate the amount of nicotine and/or alkaloids produced by various plants. The economic, health, and medicinal benefits for this technology are immediately evident. For example, tobacco having lower levels of nicotine may be produced, and nicotine production in plants not normally known for nicotine production may be produced. Alternatively, plants not normally producing nicotine may be engineered to produce it, providing an effective insecticide for those plants. It has been found that many plants express nicotine only in the green portions of the plants, but not, for example, in the fruits. According to the present invention, plants for human consumption (e.g., peppers, eggplant, tomatoes) may be engineered to produce nicotine in the leaves and stems, but not fruits, of the plant to provide a plant with an endogenously produced insecticide without resulting in high levels of nicotine to be produced in the edible portions of the plant. In a similar way, other alkaloid compounds may be produced in certain plants without concern for toxicity in humans, for example in ornamental plants. Therefore, also encompassed in the invention is the use of all or a portion of the MPO1-encoding nucleic acid to alter the production of nicotine and/or one or more alkaloid compounds in a plant cell.

The method generally comprises introducing into a cell at least one copy of an MPO1-encoding gene or a gene that encodes a non-functional MPO1 protein, and expressing the encoded protein. Where the gene encodes a functional protein, production of nicotine and/or alkaloids is increased as a result of increased conversion of precursor compounds within the relevant biosynthetic pathway, and in particular increases in production of N-methyl-pyrrolinium salt. Where the gene encodes a non-functional protein, production of nicotine and/or alkaloids is decreased as a result of decreased conversion of precursor compounds within the relevant biosynthetic pathway. In addition, where the coding region is operably linked to one or more regulable control elements, the expression of the gene can be regulated by cellular signals, which can be naturally induced or induced through manipulation of the cell's environment (e.g., by exposing the cell to one or more chemical compounds, change in temperature, change in duration of light exposure per day, wound or stress).

In another aspect, the invention provides a method of identifying substances that affect the activity of an MPO1 protein. In general, the method comprises providing an MPO1 protein, or a polypeptide comprising MPO1 enzymatic activity, exposing the protein or polypeptide to one or more substances, and determining the activity of the MPO1 protein or polypeptide. In embodiments, the method further comprises comparing the determined activity to a known activity for the protein or polypeptide in the absence of the substance(s) to determine if the activity has changed. The alteration may be an increase in activity or a decrease in activity. The method may be used to screen individual substances or many substances at once. For example, the method may be a high-throughput screening (HTS) method. The method may be used to design small molecule inhibitors against the MPO1 protein suitable for inhibiting the biosynthesis of misused products, such as cocaine. For example, a small molecule inhibitor that can be sprayed on a plant that produces cocaine and can inhibit cocaine production, has the potential to be used on known regions of cocaine production.

### EXAMPLES

The invention will be further explained by the following Examples, which are intended to be purely exemplary of the invention, and should not be considered as limiting the invention in any way.

### Example 1

### Materials and Methods

Unless otherwise indicated, the following materials and methods were used in obtaining the data discussed in the figures and Example 2.

Isolation of full length NtMPO1 cDNA and phylogenetic analysis:

Five predicted copper-containing amine oxidases (i.e. Arabidopsis thaliana, AF034579; Canavalia lineate, AF172681; Brassica juncea, AF449459; Glycine max, AF089851; and Zea mays, AY103626) were subjected to ClustalW alignment using DS Gene version 1.0 software (Accerlys Inc., San Diego, Calif.). Based upon this sequence alignment two degenerate oligonucleotides were designed and synthesized: oWGH27 (5′-GTIGTICCITAYGGIGAYCC-3′; SEQ ID NO:7) and oWGH29 (5′-GGCATIAYIGGCCARTCYTC-3′; SEQ ID NO.8), where Y is C or T; R is A or G; W is A or T; and I is inosine to reduce degeneracy (Integrated DNA Technologies, Coralville, Iowa). Eight identical PCR reactions were prepared consisting of 2 microliters (ul) of a Burley 21 root cDNA library (W. Heim, J. G. Jelesko, Plant Mol. Biol. 56:299 (2004)) and 1 micromolar (uM) of each oligonucleotide primers (oWGH27 and oWGH29) in a final reaction volume of 25 ul (F. A. Ausubel et al., Eds., Current Protocols in Molecular Biology, (John Wiley & Sons, New York, 2006). These eight PCR reactions were subjected to the following thermocycling parameters: 3 min at 94° C. for one cycle, followed by 35 cycles of denaturation at 94° C. for 1 min, annealing across a temperature gradient from 50-57° C. for 1 min, and extension at 72° C. for 1.5 min on a RoboCycler Gradient 40 (Stratagene, La Jolla, Calif.). The PCR products were then extended for 10 min at 72° C. A 10 ul aliquot of each PCR sample was separated on a 0.8% (w/v) TAE agarose gel containing 0.5 ug/ml ethidium bromide (Ausubel, above), and imaged on a Bio-Rad Gel Doc 2K System (BioRad, Hercules, Calif.). PCR products of the expected size were excised from a gel slice using the QIAEX II Gel Extraction Kit (Qiagen, Valencia, Calif.) and subcloned into pCR2.1 using the TOPO TA Cloning kit and TOP10 competent cells (Invitrogen, Carlsbad, Calif.), resulting in plasmid pWGH10. The insert in pWGH10 was fully sequenced on both strands using the Big Dye Terminator (version 3.0) Ready Reaction kit (Applied Biosystems, Foster City, Calif.) in conjunction with the oligonucleotide primers: M13 Forward; M13 Reverse; oWGH3 1, (5′-TTCACAAACTTTACGGGAGGAG-3′; SEQ ID NO.9); oWGH32, (5′-TCGAGCGAGTATCAAAGAAAT-3′; SEQ ID NO.10); oWGH33, (5′-CGTGACTGTGATCCATTCTCTGCT-3′; SEQ ID NO. 11); and oWGH34, (5′-TGTAACCCATAGATTGTGCTTCAG-3′; SEQ ID NO. 12). The cycle sequencing reactions were analyzed at the Core Laboratory Facility at the Virginia Bioinformatics Institute (Virginia Polytechnic Institute and State University, Blacksburg, Va.) using an ABI 3100 (Applied Biosystems) capillary sequencer. The resulting DNA trace files were assembled into contigs and edited using the SeqMan Windows 32 version 5.07 in the Lasergene software package (DNASTAR, Madison, Wis.). The genomic DNA blot analysis was performed with B21 genomic DNA and hybridized using high stringency conditions to a dUTP-digoxygenin-labeled PCR fragment amplified using oJGJ156 (5′-TCCATGGCCACTACTAAACAGAAAG-3′; SEQ ID NO. 13) and oJGJ179 (5′-TAACAGGCCAGTCTTCCAACCGAG-3′; SEQ ID NO. 14) using pWGH15 as template DNA.

Plasmid pWGH10DNA and oligonucleotide primers oWGH27 and oWGH29 were used to generate a PCR amplified Digoxygenin-dUTP-labeled DNA fragment that was used as a hybridization probe for screening a B21 root cDNA phagmid library using the same methods as previously described in Heim et al., above. This resulted in the isolation of pWGH15 containing an approximate 2.8 Kb cDNA insert. Plasmid pWGH15 was randomly mutagenized with the GeneJumper transposon (Invitrogen) to introduce novel oligonucleotide priming sites that facilitated complete DNA sequencing of the insert. The nucleotide sequences of pWGH10 and pWGH15 were aligned using CLUSTALW in DS GENE version 1.5 (Accelrys Inc). The predicted protein sequences encoded by these two plasmids were also aligned with four amine oxidase proteins for which X-ray crystal structures have been solved (M. R. Parsons et al., Structure 3, 1171 (1995); R. Li, J. P. Klinman, F. S. Mathews, Structure 6, 293 (1998);V. Kumar et al., Structure 4, 943 (1996); M. C. Wilce et al., Biochemistry 36, 16116 (1997)). BLASTN and BLASTX searches on non-redundant Genbank databases were also performed using DS GENE. Predicted proteins identified in the BLASTX analysis were aligned using CLUSTALW and a phylogenetic tree was generated using the Neighbor Joining method utilizing Poisson Correction with 1000 iterations of Bootstrap analysis in DS Gene. Small subunit ribosomal RNA sequences from various species were aligned by CLUSTALW in DS GENE and then subjected to maximum likelihood analysis in PAUP version 4.0 Beta for Windows (Sinauer Associates, Inc., Sunderland, Mass.) the ribosomal RNA-based tree was generated with TreeView (Win32) version 1.6.6. Dot plot comparison of symbiotic islands was performed using DS Gene version 1.5. The R. palustris and B. japonicum whole bacterial genome sequence alignments were performed using MAUVE (http://gel.ahabs.wisc.edu/mauve/) (A. C. E. Darling, B. Mau, F. R. Blattner, N. T. Pema, Genome Res. 14, 1394 (2004)).

Primary root cultures and mRNA expression analysis: B21 and LA21 primary root cultures were grown and RNA extracted as previously described in Heim et al., above. The same pWGH10 digoxygenin-dUTP labeled probe that was used for screening the cDNA library was also used to monitor the steady state mRNA levels of NtDAO1-like genes. Hybridization with a β-ATPase digoxygenin-dUTP labeled PCR fragment (D. G. Reed, J. G. Jelesko, Plant Sci. 167, 1123 (2004)) was used to examine the steady state mRNA levels of a housekeeping gene that does not change during these conditions (D. G. Reed, J. G. Jelesko, Plant Sci. 167, 1123 (2004); D. E. Riechers, M. P. Timko, Plant Mol. Biol. 41, 387 (1999); B. Xu, M. J. Sheehan, M. P. Timko, Plant Growth Regulation 44, 101 (2004); W. G. Heim, R.-H. Lu, J. G. Jelesko, Plant Sci 170, 835 (2006)). Quantitative Real Time PCR was performed on B21 and LA21 root RNA using oligonucleotide primers oJGJ178 (5′-TCAAAATCCCCGTGTTGGCGAG-3′; SEQ ID NO. 15) and oJGJ179 using pWGH15 to generate a standard curve, as previously described (S. K. Kidd et al., Plant Mol Biol 6, 699 (2006).

Assay of recombinant NtDAO1 in bacteria: In order to facilitate the cloning of the NtDAO1 gene into a recombinant expression vector Ba HI and NcoI sites were introduced upstream of the predicted ATG start codon, using oligonucleotide primers oJGJ166 (5′-GGATCCCCATGGCCACTACTAAACAGAAAG-3′; SEQ ID NO.16) and oJGJ157 (5′-TGGTAGAGGTATTGGTGGAAAG-3′; SEQ ID NO.17) to amplify a 241 bp PCR fragment using pWGH 15 as template DNA. This modified fragment was cloned into pCR2.1 (Invitrogen) to yield pJGJ367. A 165 bp BamHI-SalI fragment was cut from pJGJ367 and ligated into pWGH15 similarly cut, resulting in pJGJ369. Finally, a 2.6 Kb NcoI-XhoI (partial) fragment was cut from pJGJ369 and ligated into pET32a+ similarly cut, to yield pJGJ389. Plasmid pJGJ389 was transformed into the Rosetta E. coli strain (Novagen, Madison, Wis.) for expression of a recombinant TRX-NtDAO1 protein. Mid-log phase Rosetta/pJGJ389 cells were cultured overnight in LB media supplemented with 100 ug/ml Ampicillin, 30 ug/ml Chloramphenicol, and 0.2 mM isopropyl-β-D-thiogalactoside at 18° C. at 250 rpm. The cells were pelleted, lysed, and the native protein extract incubated with Ni-NTA superflow resin, and the TRX-MPO1 was eluted as per manufacture's instructions (Qiagen, Valencia, Calif.). The TRX-MPO1 enriched extract was mixed 1:1 (v:v) with 100% glycerol and stored at −20° C. Prior to use, the recombinant protein extracts were buffer exchanged using a PD-10 column (GE Health Care Bio-Sciences AB, Uppsala, Sweden) into the same buffer used for the subsequent spectrophotometric-based amine oxidase assay (Kusche, W. Lorenz, in Methods of Enzymology, H. U. Bergmeyer, Ed. (Verlag Chemie, Weinheim, GmbH, 1983), vol. 3, pp. 237-250) on a Beckman DU-7400 spectrophotometer (Beckman Coulter Inc., Fullerton, Calif.). A variety of diamine substrates were assayed: putrescine, cadaverine, 1,3-diaminepropane (Sigma-Aldrich Co., St. Louis, Mo.), and N-methyl-1,4 diaminebutane (Toronto Research Chemicals Inc, North York, ON, Canada). Background-corrected diamine oxidase rates were graphed as Lineweaver-Burk plots in order to estimate the Vmax and Km of the recombinant TRX -NtMPO1 fusion protein isolated from three independent extracts. A General Linear Model ANOVA test was performed using Minitab version 14 for Windows (Minitab Inc., State College, Pa.) to determine whether the kinetic properties of each substrate were significantly different from those observed with N-methylputrescine as substrate.

### Example 2

### Cloning and Analysis of a Tobacco cDNA Encoding MPO and Analysis of the MPO

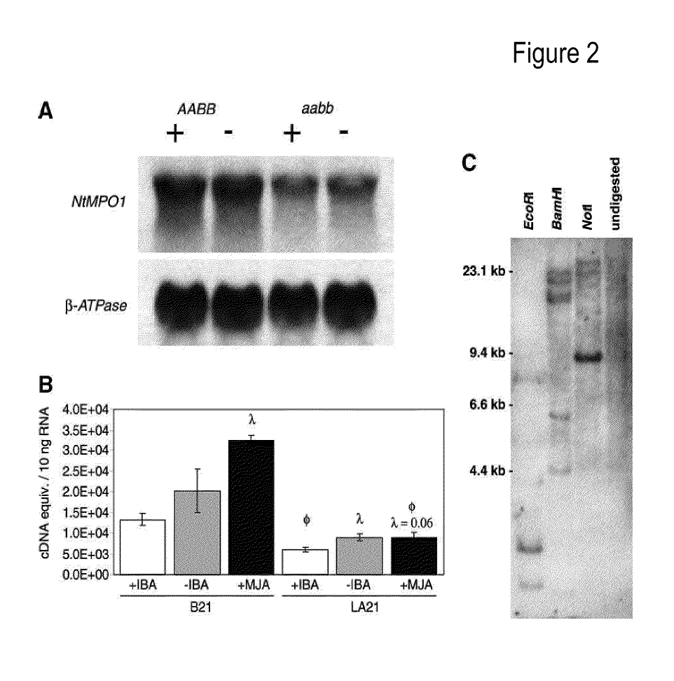

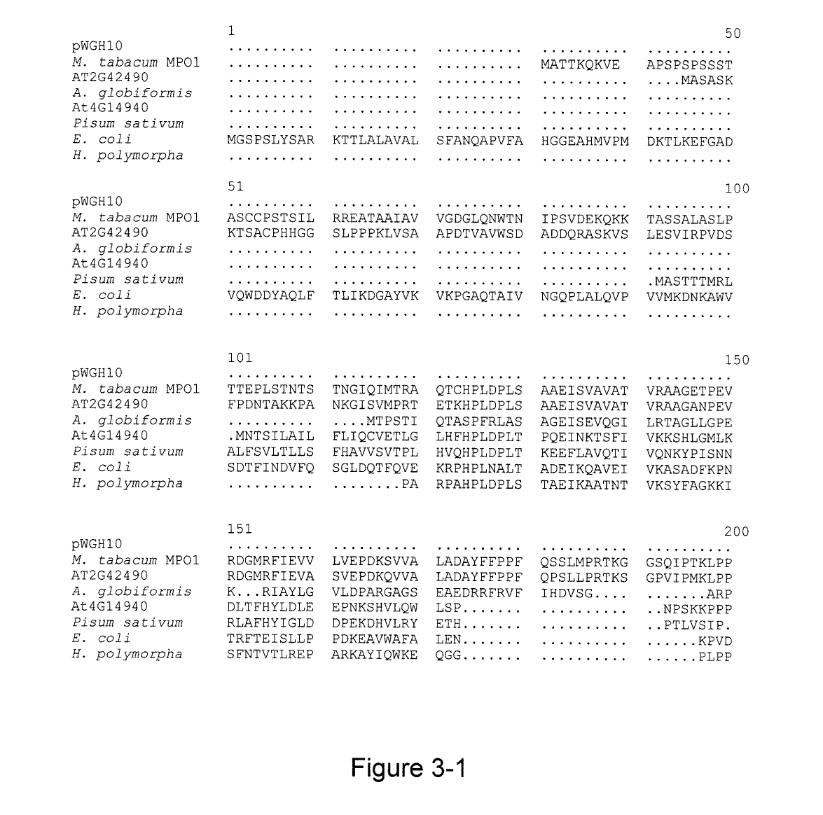

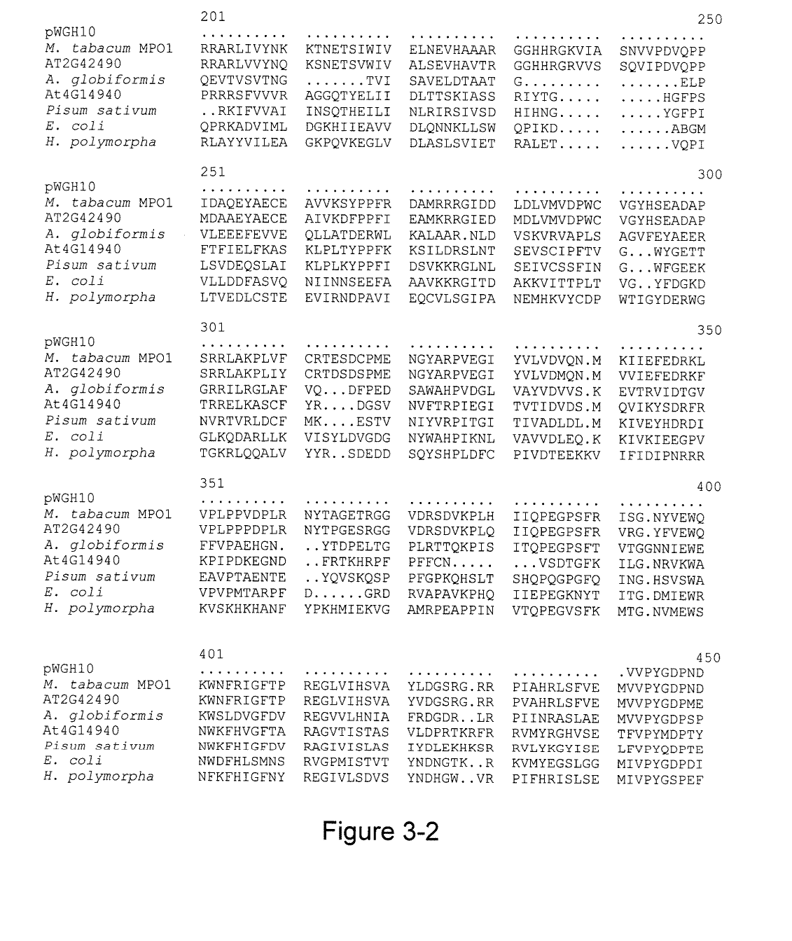

We utilized a degenerate oligonucleotide strategy to amplify a gene fragment encoding a putative Nicotiana tabacum MPO enzyme. This resulted in the cloning of a 986 bp PCR fragment (i.e., plasmid pWGH10) amplified from a Burley 21 root cDNA library. BLASTX analysis of this DNA fragment showed highest similarity (E-value=1×10⁻¹⁷³) to an Arabidopsis gene (At2G42490) encoding a copper-containing amine oxidase belonging to the same enzyme class as tobacco MPO (EC1.4.3.6). To determine if the corresponding N. tabacum gene was subject to genetic regulation by the A and B loci, axenic Burley 21 roots (B21, with wild type AABB genotype) and Low-Alkaloid Burley 21 roots (LA21, with the double mutant aabb genotype) were grown either in media resulting in low expression of nicotine biosynthetic genes or media that increases expression of nicotine biosynthetic genes (10, 13). Total RNA was isolated from these primary root cultures and subjected to RNA blot analysis. FIG. 2A shows that the MPO-like transcript levels in the mutant LA21 roots were lower than those in B21 roots, indicating the MPO-like transcript levels were regulated by the A and B loci. The pWGH10 insert was used as a hybridization probe to isolate plasmid pWGH15, containing a 2.8 Kb cDNA. Plasmid pWGH15 was used to design oligonucleotide primers that were used in QRT-PCR analysis of RNA from B21 and LA21 root cultures that were treated with two different conditions (i.e., IBA deprivation and methylj asmonic acid (MJA) treatment) that induce nicotine biosynthetic genes (11, 12, 14). FIG. 2B shows that transcript levels were significantly reduced in the mutant LA21 roots during control and MJA treatments. Thus, MPO-like transcript accumulation levels were regulated by the A and B loci and increased during conditions that enhance expression of known nicotine biosynthetic genes.

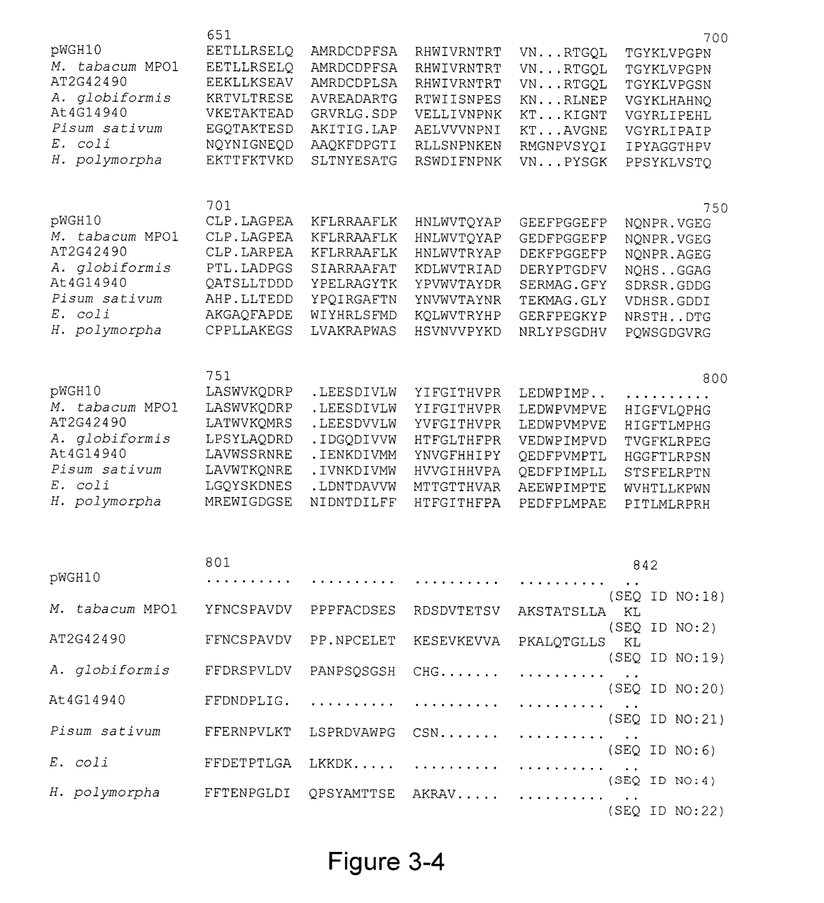

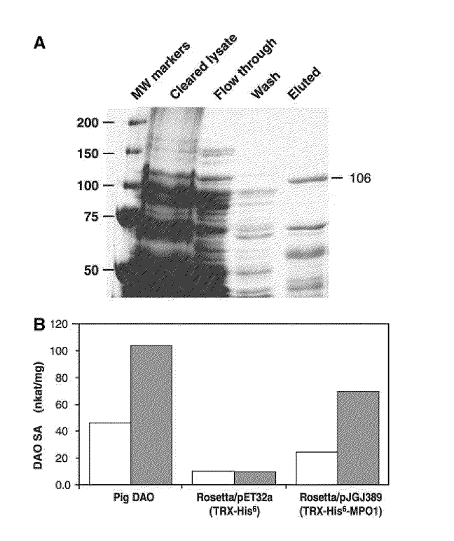

The insert in pWGH10 was 98.6% similar to the corresponding region in pWGH15, suggesting they represent a multigene family (FIG. 3). DNA blot analysis of B21 genomic DNA indicated the presence of a small multigene family of approximately six members (FIG. 2C). The assigned ATG start codon was preceded by stop codons in all reading frames, suggesting pWGH15 was a full-length cDNA encoding a 790 amino acid polypeptide. BLASTX analysis with the pWGH15 DNA sequence showed low E-values with many other predicted copper amine oxidase proteins (data not shown). Based upon the predicted class of enzyme and mRNA expression patterns, this gene was tentatively named NtMPO1. The predicted NtMPO1 protein sequence was aligned with four copper amine oxidases for which X-ray crystal structures are available. The highly conserved Asp-Tyr⁵⁰⁹-Glu/X motif, containing the tyrosine that is post-translationally oxidized by a copper ion into a topaquinone (15, 16) forming part of the catalytic site, was conserved in the predicted NtMPO1 protein, as well as three histidines that are responsible for coordinating a copper ion near the reactive tyrosine⁵⁰⁹/topquinone⁵⁰⁹ (FIG. 3). The NtMPO1 cDNA was subcloned into pET32a+ and a 106 kDa recombinant thioredoxin-NtMPO1 (TRX-NtMPO1) fusion protein was enriched from bacterial extracts using metal binding chromatography (FIG. 4A). The TRX-NtMPO1 extracts showed substrate-specific amine oxidase activity over background levels (FIG. 4B). The recombinant TRX-MPO1 oxidatively deaminated N-methylputresine, putrescine, cadaverine, and 1,3-diaminepropane, but with different kinetics for each substrate (Table 1).

TABLE 1
Enzyme kinetics of recombinant TRX-MPO protein

| Substrate | Vₘₐₓ (mM min⁻¹) | Kₘ (mM) | Vₘₐₓ/Kₘ (min⁻¹) |
|---|---|---|---|
| N-methylputrescine | 0.0017 ± 0.0001 | 0.19 ± 0.02 | 87.2 × 10⁻⁴ |
| putrescine | 0.0007 ± 0.0001 | 0.76 ± 0.16 | 8.7 × 10⁻⁴ |
| | (P = 0.0003) | (P = 0.0156) | |
| cadaverine | 0.0003 ± 0.00004 | 1.79 ± 0.16 | 1.7 × 10⁻⁴ |
| | (P < 0.0001) | (P < 0.0001) | |
| 1,3-diamine propane | 0.0007 ± 0.0001 | 0.35 ± 0.03 | 19.3 × 10⁻⁴ |
| | (P = 0.0003) | (P = 0.7021) | |

The Vₘₐₓ/Kₘ ratios confirmed that the recombinant TRX-NtMPO1 preferred N-methylputrescine as substrate. These results are in good agreement with previous reports of MPO enzyme activity from plant extracts (8, 17). Thus, plasmid pWGH15 encodes a bona fide MPO activity from Nicotiana tabacum that contributes to nicotine biosynthesis.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

### REFERENCES CITED

The following references are cited above by reference to their respective number. These references, as well as those specifically cited above by citation, are incorporated herein by reference.
1. R. F. Dawson, Am J Bot 29, 813 (1942).
2. F. Saitoh, M. Mona, N. Kawashima, Phytochemistry 24, 477 (1985).
3. I. T. Baldwin, J. Chem. Ecol. 15, 1661 (1989).
4. I. T. Baldwin, Proc Natl Acad Sci USA 95, 8113 (1998).
5. M. Tomizawa, J. E. Casida, Annu Rev Entomol 48, 339 (2003).
6. S. J. Sinclair, K. J. Murphy, C. D. Birch, J. D. Hamill, Plant Mol. Biol. 44, 603 (2000).
7. J. Q. Saunders, L. P. Bush, Plant Physiol. 64, 236 (1979).
8. S. Mizusaki, Y. Tanabe, M. Noguchi, E. Tamaki, Phytochemistry 11, 2757 (1972).
9. P. D. Legg, J. F. Chaplin, G. B. Collins, J. Hered. 60, 213 (1969).
10. N. Hibi, S. Higashiguchi, T. Hashimoto, Y. Yamada, Plant Cell 6, 723 (1994).
11. K. A. Cane, M. Mayer, A. J. Lidgett, A. J. Michael, J. D. Hamill, Functional Plant Biology 32, 305 (2005).
12. S. K. Kidd et al., Plant Mol Biol 6, 699 (2006).
13. D. G. Reed, J. G. Jelesko, Plant Sci. 167, 1123 (2004).
14. T. Shoji, Y. Yamada, T. Hashimoto, Plant Cell Physiol. 41, 831 (2000).
15. R. Matsuzaki, T. Fukui, H. Sato, Y. Ozaki, K. Tanizawa, FEBS Lett 351, 360 (1994).
16. K. Tanizawa, R. Matsuzaki, E. Shimizu, T. Yorifuji, T. Fukui, Biochem Biophys Res Commun 199, 1096 (1994).
17. T. Hashimoto, A. Mitani, Y. Yamada, Plant Physiol. 93, 216 (1990).
18. U. Dobrindt, B. Hochhut, U. Hentschel, J. Hacker, Nat Rev Microbiol 2, 414 (2004).
19. T. Kaneko et al., DNA Res 7, 331 (2000).
20. J. T. Sullivan et al., J Bacteriol 184, 3086 (2002).
21. J. T. Sullivan, C. W. Ronson, Proc Natl Acad Sci USA 95, 5145 (1998).
22. Y. Anzai, H. Kim, J. Y. Park, H. Wakabayashi, H. Oyaizu, Int J Syst Evol Microbiol 50, 1563 (2000).
23. T. Kaneko et al., DNA Res 9, 189 (2002).
24. P. J. Christie, K. Atmakuri, V. Krishnamoorthy, S. Jakubowski, E. Cascales, Annu Rev Microbiol 59, 451 (2005).
25. A. Hubber, A. C. Vergunst, J. T. Sullivan, P. J. Hooykaas, C. W. Ronson, Mol Microbiol 54, 561 (2004).
26. J. G. Jelesko, K. Carter, Y. Kinoshita, W. Gruissem, Mol Genet Genomics 274, 91 (2005).
27. D. Chantal, M.-D. Chilton, J. Tempe, Bio/technology 2, 73 (1984).
28. D. Tepfer, Cell 37, 959 (1984).
29. S. Aoki, J Plant Res 117, 329 (2004).
30. D. Valvekens, M. V. Montagu, M. V. Lijsebettens, Proc Natl Acad Sci USA 85, 5536 (1988).
31. Y. Boucher, W. F. Doolittle, Mol Microbiol 37, 703 (2000).
32. B. M. Lange, T. Rujan, W. Martin, R. Croteau, Proc Natl Acad Sci USA 97,13172 (2000).

1. An isolated or purified nucleic acid comprising the coding sequence for an N-methylputrescine oxidase (MPO) protein.

2. The nucleic acid of claim 1, wherein the nucleic acid comprises the coding sequence for an MPO protein from Nicotiana tabacum.

3. The nucleic acid of claim 1, wherein the nucleic acid comprises the sequence of SEQ ID NO:1.

4. A vector comprising the nucleic acid of claim 1.

5. A recombinant cell comprising the nucleic acid of claim 1.

6. The cell of claim 5, wherein the cell is a plant cell.

7. The cell of claim 5, wherein the cell comprises two or more nucleic acid sequences encoding an MPO protein.

8. The cell of claim 5, which comprises elevated levels of an MPO protein, as compared to a non-recombinant, but otherwise identical, cell.

9. A recombinant cell comprising a defective nucleotide sequence encoding an MPO protein, wherein the defective sequence comprises a deletion, addition, or mutation of one or more nucleotides of an MPO gene of the cell.

10. The cell of claim 9, which comprises reduced levels of an MPO protein, as compared to a non-recombinant, but otherwise identical, cell.

11. An isolated, purified, or recombinant polypeptide comprising an amino acid sequence that provides MPO activity when properly folded.

12. The polypeptide of claim 11, wherein the amino acid sequence comprises a sequence for an MPO protein from Nicotiana tabacum.

13. The polypeptide of claim 12, wherein the polypeptide comprises the sequence of SEQ ID NO:2.

14. A host cell comprising the polypeptide of claim 11.

15. The cell of claim 11, wherein the cell is a plant cell.

16. A method of altering the production of nicotine and/or one or more alkaloid compounds in a plant cell, said method comprising:
introducing into a cell at least one copy of an MPO1-encoding gene or a gene that encodes a non-functional MPO1 protein; and
expressing the encoded protein.

17. The method of claim 16, wherein expressing the encoded protein is by way of regulated expression.

18. A method of identifying substances that affect the activity of an MPO1 protein; said method comprising:
providing an MPO1 protein, or a polypeptide comprising MPO1 activity;
exposing the protein or polypeptide to one or more substances; and
determining the activity of the MPO1 protein or polypeptide.

19. The method of claim 18, further comprising:
comparing the determined activity to a known activity for the protein or polypeptide in the absence of the substance(s) to determine if the activity has changed.

20. The method of claim 19, wherein the method is a high-throughput screening (HTS) method.

21. A method of producing transgenic proteins in a low nicotine plant, said method comprising:
integrating into a cellular genome of a cell at least one copy of a defective MPO1-encoding gene, wherein integrating results in reduction or abolition of production of nicotine by the cell;
introducing into the cell at least one copy of a second gene which encodes a desired protein; and
expressing the desired protein.
